(19) United States
(12) Patent Application Publication
(10) Pub. No.: US 2018/0168855 A1
(43) Pub. Date: Jun. 21, 2018

(54) JOINT COMPRESS COLD PACK

(71) Applicant: Penguin Fingers, LLC, Las Vegas, NV (US)

(72) Inventors: Benjamin Troy Landen, Las Vegas, NV (US); Prescott Foster Bailey, JR., Las Vegas, NV (US)

(21) Appl. No.: 15/842,509

(22) Filed: Dec. 14, 2017

Related U.S. Application Data
(60) Provisional application No. 62/434,515, filed on Dec. 15, 2016.

(51) Int. Cl. A61F 7/02

(52) U.S. Cl. CPC A61F 7/02; A61F 2007/0037; A61F 2007/0219

(57) ABSTRACT

A joint compression cold pack and methods of using the same are disclosed. A cold pack comprises a cylindrical outer casing, a liquid comprising an anti-freeze material and water encased within the cylindrical outer casing, and an aperture formed in a middle of the cylindrical outer casing filled with the liquid, wherein the aperture accepts a selected body part of a patient. A method for using the cold pack comprises freezing the cold pack for a predetermined period of time, and accepting, through the aperture of the cylindrical casing, a selected body part of a patient.

### CROSS-REFERENCE TO RELATED APPLICATIONS

This application claims priority to U.S. Provisional Patent Application Ser. No. 62/434,515, filed Dec. 15, 2016, the entire contents of which is herein incorporated by reference.

### FIELD OF THE DISCLOSURE

The present invention relates to cold packs and, more particularly, to a joint compress cold pack.

### BACKGROUND

Cold packs and compression devices have long been used to treat minor injuries of various body parts, such as sprains, swelling, and mild burns. However, current cold packs or other compression devices are difficult to use with small joints, such as finger and toe joints, and/or other joints that are typically hard to reach or difficult to keep cold pack in place due to size and/or movement of the joint. Moreover, traditionally, cold packs do not provide compression specifically to the joint. Additionally, current cold packs are often too large or too small, such that they provide too much compression and/or cold therapy on body parts where it is not needed, or not enough compression and/or cold therapy directly where it is needed, such as a joint. The size of the cold pack, or its inability to stay in place on the desired body part, also frequently limits movement of the patient unnecessarily during therapy.

Many cold packs contain latex, to which some patients are allergic, or contain toxins or other dangerous and/or undesirable chemicals, such as polyvinyl chloride (PVC), bisphenol A (BPA), bisphenol A (BPS), phthalates, formaldehyde, perfluorocarbons (PFCs), 2-(2-Methoxyethoxy)ethanol (DEGME), ethylene glycol monobutyl ether (EGBE), Bis(2-ethylhexyl) phthalates (DEHPs), and dioxins. Finally, many cold packs are intended for only one use and may not be refrozen once used.

The present invention is aimed at one or more of the problems identified above.

### BRIEF SUMMARY OF THE INVENTION

In one embodiment, a cold pack is provided. A cold pack comprises a cylindrical outer casing, a liquid comprising an anti-freeze material and water encased within the cylindrical outer casing, and an aperture formed in a middle of the cylindrical outer casing filled with the liquid, wherein the aperture accepts a selected body part of a patient.

In another embodiment, method for using a cold pack is provided. The method comprises a first step of freezing the cold pack for a predetermined period of time, wherein the cold pack comprises: a cylindrical outer casing, a liquid comprising an anti-freeze material and water encased within the cylindrical outer casing, and an aperture formed in a middle of the cylindrical outer casing filled with the liquid. The method comprises a second step of accepting, through the aperture of the cylindrical casing, a selected body part of a patient.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a front perspective view of a joint compress cold pack.

FIG. 2 is a side view of the joint compress cold pack.

FIG. 3 is a top view of the joint compress cold pack.

FIG. 4 is front view of the joint compress cold pack of FIGS. 1-3, illustrated on a human finger joint.

FIG. 5 is top view of the joint compress cold pack of FIGS. 1-3, illustrated on a human finger joint.

FIG. 6 is a flowchart illustrating a method for using the joint compress cold pack of FIGS. 1-3.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

Reference throughout this specification to “one embodiment”, “an embodiment”, “one example” or “an example” means that a particular feature, structure or characteristic described in connection with the embodiment or example is included in at least one embodiment of the present invention. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

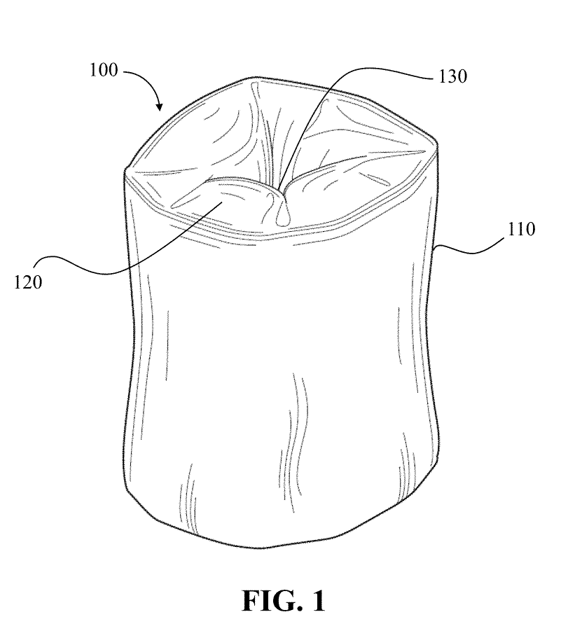

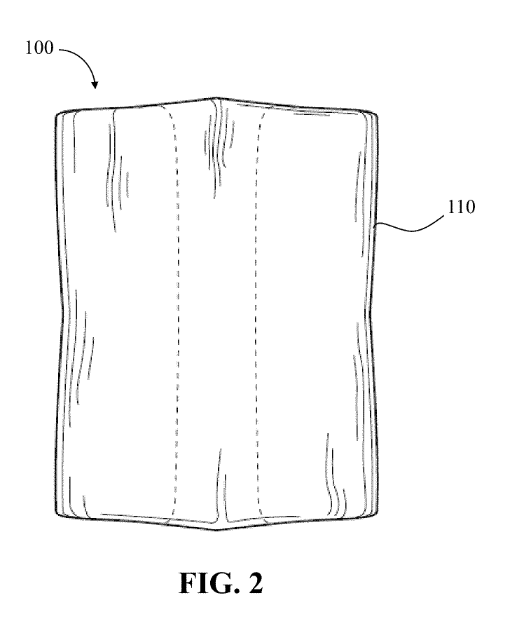

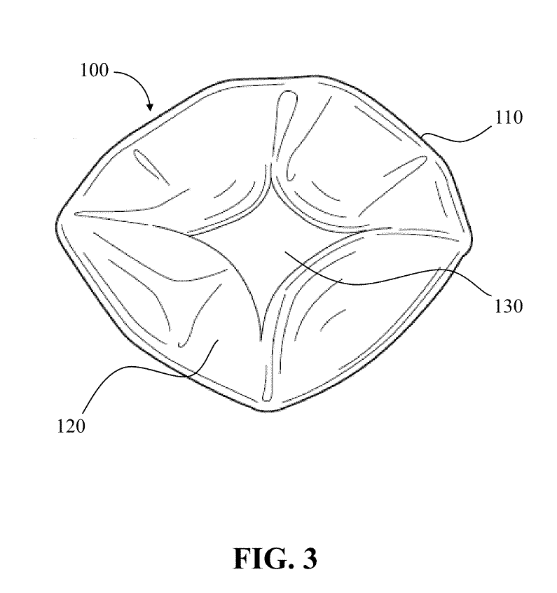

The present invention provides a joint compress cold pack 100. Referring now to FIGS. 1-3, a front perspective view, a side view, and a top view of the joint compress cold pack 100 are illustrated, respectively. The joint compress cold pack 100 provides 360 degree cold compress therapy for a single joint to alleviate symptoms from, for example, joint swelling, arthritis, tendonitis, burns, and injury.

The joint compress cold pack 100 comprises a flexible outer casing 110. The outer casing 110 may comprise a tube/cylindrical casing, the inside of which is filled with a mixture of water and an anti-freeze material 120. The anti-freeze material 120 may comprise a liquid, solid, or semisolid (such as a gel). The anti-freeze material 120 may comprise propylene glycol or other type of anti-freezing agent, that may become cold when placed in a refrigerator or freezer, but will never freeze completely. An aperture 130 is formed in the middle of the outer casing 100.

The outer casing 110 may be comprised of any material of suitable flexibility and durability. By way of example and not limitation, the outer casing 110 may comprise thermoplastic rubber (TPR), thermoplastic elastomers (TPE), including, for example, thermoplastic polyurethane (TPU), thermoplastic olifins (TPE-o), styrenicblock copolymers (TPE-s), thermoplastic copolyesters, thermoplastic copolyamides, thermoplastic vulcanisates (TPE-v or TPV), or elastomertic alloys.

In one embodiment, the joint compress cold pack 100 may optionally comprise a secondary casing (not shown) around the outer casing 110. The secondary casing may comprise a thin material, such as spandex, for additional comfort (e.g., to protect the skin from the cold outer casing 110).

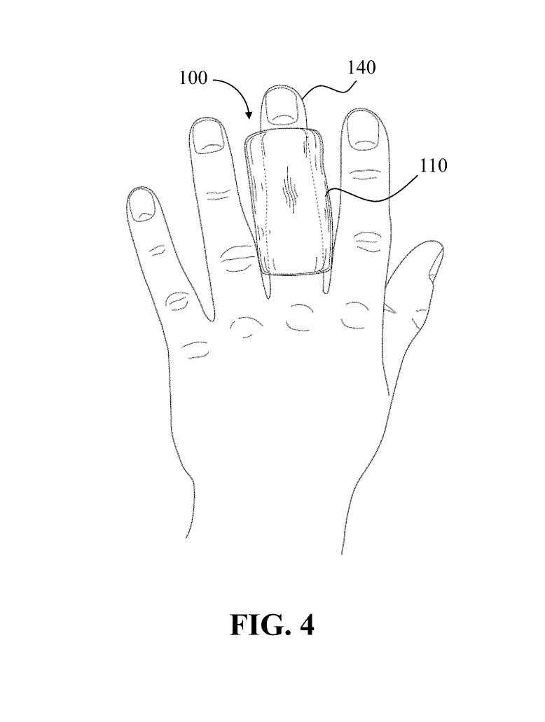

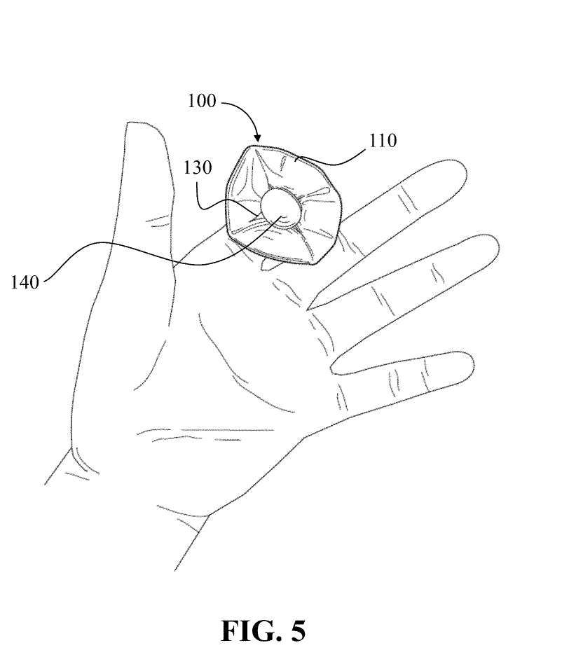

The joint compress cold pack 100 may be of any size to comfortably fit on a joint. Referring now to FIGS. 4-5, a front view and a top view of the joint compress cold pack of FIGS. 1-3 are illustrated on a human finger joint. The elasticity of the outer casing 110 filled with the anti-freeze material 120 allows for a body part 140 (such as a finger, as illustrated) to be placed through the aperture 130 of the outer casing 100. Once applied to the body part 140, the joint compress cold pack 100 compresses the area of the finger that is placed into the aperture 130 of the outer casing 110 (for instance, a finger joint, as illustrated), and can optionally provide cold therapy if the joint compress cold pack 100 is refrigerated or frozen.

According to illustrated embodiment, the joint compress cold pack 100 may be about 1 to 2 inches wide, 2 to 3 inches long, and 1 to 2 inches in depth, such that it fits comfortably on most adult fingers and toes. However, it will be understood that the joint compress cold pack 100 may be different sizes for different applications other than for use on human fingers, such as for other body parts that include joints, including toes, wrists, ankles, knees, and elbows, and/or for body parts that require 360-degree therapy, such as parts of the legs and arms, and/or any other suitable body part. For example, according to an alternate embodiment, the joint compress cold pack 100 may be about 4 inches wide and 4 inches long for use on a wrist. In another embodiment, the joint compress cold pack 100 may be about 6 inches wide and 6 inches long for use on an elbow. In yet another embodiment, the joint compress cold pack 100 may be about 8 inches wide and 8 inches long for use on a knee. In yet another embodiment, the joint compress cold pack 100 may be about 12 inches wide and 8 inches long for use on a shin.

In other embodiments, the joint compress cold pack 100 may be different sizes based on the intended patient. For example, the joint compress cold pack 100 may be smaller to fit on smaller adults or children, or the joint compress cold pack 100 may be larger to fit on larger adults. It is envisioned that the joint compress cold pack 100 could also be used for non-human applications, such as veterinary applications, wherein the size of the joint compress cold pack 100 would be tailored to the specific intended use.

The joint compress cold pack 100 of the present invention provides several advantages over the prior art. First, the joint compress cold pack 100 provides compression to joints that are typically hard to reach or difficult to keep a cold pack in place due to size and/or movement of the joint. Second, whereas traditional cold pack are often too large or too small, such that they provide too much or not enough compression and/or cold therapy, the joint compress cold pack 100 can be tailored to any size based on the target joint. Third, the composition of the joint compress cold pack 100 creates an easy, roll-on application, and the joint compress cold pack 100 stays in place once applied to the joint. Fourth, the joint compress cold pack 100 may be comprised of latex-free and non-toxic materials and fillers, and does not require the use of dangerous and/or undesirable chemicals, such as polyvinyl chloride (PVC), bisphenol A (BPA), bisphenol A (BPS), phthalates, formaldehyde, perfluorocarbons (PFCs), 2-(2-Methoxyethoxy)ethanol (DEGME), ethylene glycol monobutyl ether (EGBE), Bis(2-ethylhexyl) phthalates (DEHPs), and dioxins. Finally, the joint compress cold pack 100 is reusable and, if desired, may be refrozen.

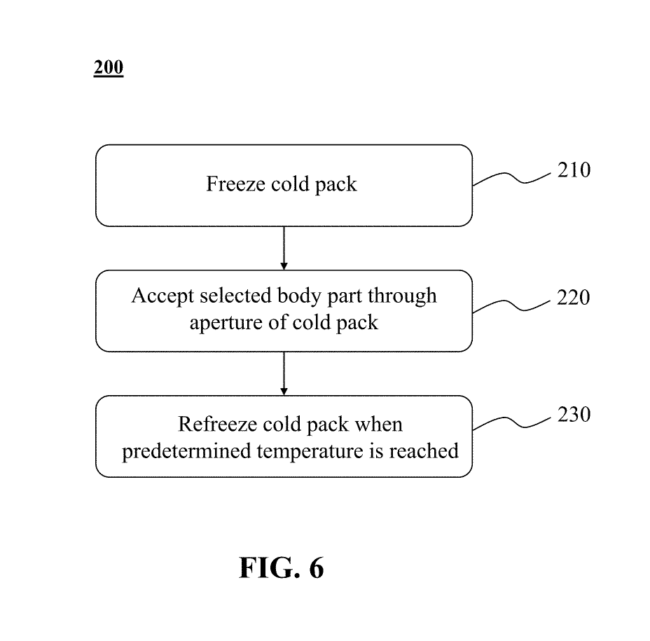

Referring now to FIG. 6, a flowchart illustrating a method 200 for using the joint compress cold pack 100 of FIGS. 1-3 is shown. At a first step 210, the joint compress cold pack 100 is frozen, by, for example, the user placing the joint compress cold pack 100 in a freezer for a given amount of time. At a second step 220, the joint compress cold pack 100 accepts, through the aperture 130 of outer casing 110, the selected body part 140 of a patient. As previously mentioned, the body part 140 may include a joint, such as a finger, toe, wrist, ankle, knee, or elbow, or may simply be a body part that requires 360-degree therapy, such as parts of legs or arms. At a third step 230, the joint compress cold pack 100 may be refrozen when a temperature of the joint compress cold pack 100 reaches a predetermined temperature, such as room temperature, and refrozen. In some embodiments, the joint compress cold pack 100 may include markings, temperature-dependent coloring, or other temperature indicators to signal to the patient when the predetermined temperature has been reached.

The above description of illustrated examples of the present invention, including what is described in the Abstract, are not intended to be exhaustive or to be limitation to the precise forms disclosed. While specific embodiments of, and examples for, the present invention are described herein for illustrative purposes, various equivalent modifications are possible without departing from the broader spirit and scope of the present invention. Indeed, it is appreciated that the specific measurements, materials, etc., are provided for explanation purposes and that other values may also be employed in other embodiments and examples in accordance with the teachings of the present invention.

What is claimed is:

1. A cold pack comprising:
a cylindrical outer casing;
a liquid comprising an anti-freeze material and water encased within the cylindrical outer casing; and
an aperture formed in a middle of the cylindrical outer casing filled with the liquid, wherein the aperture accepts a selected body part of a patient.

2. The cold pack of claim 1, wherein the cylindrical outer casing is comprised of a flexible material.

3. The cold pack of claim 2, wherein the flexible material comprises one of a thermoplastic rubber and a thermoplastic elastomer.

4. The cold pack of claim 1, wherein the anti-freeze material comprises propylene glycol.

5. A method for using a cold pack, the method comprising:
freezing the cold pack for a predetermined period of time, wherein the cold pack comprises: a cylindrical outer casing, a liquid comprising an anti-freeze material and water encased within the cylindrical outer casing, and an aperture formed in a middle of the cylindrical outer casing filled with the liquid; and
accepting, through the aperture of the cylindrical casing, a selected body part of a patient.

6. The method of claim 5, further comprising:
refreezing the cold pack when the cold pack reaches a predetermined temperature.

7. The method of claim 5, wherein the cylindrical outer casing is comprised of a flexible material.

8. The method of claim 7, wherein the flexible material comprises one of a thermoplastic rubber and a thermoplastic elastomer.

9. The method of claim 5, wherein the anti-freeze material comprises propylene glycol.

10. The method of claim 5, wherein the cold pack further comprises a temperature indicator to signal when the predetermined temperature is reached.

11. The method of claim 10, wherein the temperature indicator includes one of a marking or a coloring.
